(19) United States
(12) Patent Application Publication
(10) Pub. No.: US 2007/0196815 A1
(43) Pub. Date: Aug. 23, 2007

(54) Positive Selection Procedure for Optically Directed Selection of Cells

(76) Inventors: Jason Lappe, Mesa, AZ (US); Neal Woodbury, Tempe, AZ (US); Carole Flores, Mesa, AZ (US)

Correspondence Address: NEEDLE & ROSENBERG, P.C., SUITE 1000, 999 PEACHTREE STREET, ATLANTA, GA, 30309-3915, US

(21) Appl. No.: 11/687,722

(22) Filed: Mar. 19, 2007

Related U.S. Application Data
(63) Continuation-in-part of application No. 10/343,412, filed on Jan. 28, 2003, now Pat. No. 7,193,706.
(60) Provisional application No. 60/222,691, filed on Aug. 2, 2000.

(51) Int. Cl. C12Q 1/00; G06T 7/00

(52) U.S. Cl. 435/4; 382/120

(57) ABSTRACT

Provided are methods and systems that can be used as a means of positive selection by initiating light mediated DNA repair in cells, having photosensitive repair systems, with desired characteristics from libraries of mutants that have been deactivated with a lethal light source.

### CROSS REFERENCE TO RELATED APPLICATION

This application is a Continuation-in-Part of U.S. Utility application Ser. No. 10/343,412, filed Aug. 7, 2003, which in turn claims priority to International Application No. PCT/US01/24365, filed Aug. 2, 2001, which in turn claims priority to U.S. Provisional Application No. 60/222,691, filed Aug. 2, 2000, which are herein incorporated by reference in their entirety.

### BACKGROUND

Directed evolution is a process wherein the sequence of a gene is varied randomly by any of a number of methods, generating a library of mutated genes.

These mutated genes are expressed and the functions of those gene products are assayed. A selection or screening procedure is then applied to select those cells containing genes that express products-with desirable functions. These cells, and their genes, are then selectively amplified, and the mutagenesis, screening and selection process is repeated until gene products with the most desirable functions are obtained.

The general scheme for directed evolution is shown in FIG. 1. First, variation is introduced into the gene in question by some type of random mutagenesis and a library of sequences is introduced into an organism (typically organisms such as Escherichia coli or yeast, though other types of cell cultures can be used) for expression of the altered proteins. Next, this population of cells is screened for the desired activity and individual colonies are selected. Finally, these selected cells are grown up (amplification of the selected genetic variants) and the plasmids expressing proteins with the most desirable functional traits are isolated. These then are used as heterogeneous templates for further random mutagenesis and reintroduced into the cells for another round of screening and amplification. This cycle is continued until the desired functional characteristics are achieved.

Directed evolution has been successfully used to generate new molecules with altered physical or chemical characteristics. For example, Doi et al. modified green fluorescent protein (GFP) to include a binding site for the TEM1-lactamase inhibitor and then used directed evolution methods to produce a protein molecule whose fluorescent properties changed upon binding the target molecule. Directed evolution methodologies involving fluorescent proteins are particularly useful for the development of sensors, tags or probe systems, particularly for in vivo applications.

GFP is one of a few different proteins that, in the absence of any externally supplied cofactor, fluoresces strongly in the visible region of the spectrum. A number of these proteins including GFP and numerous engineered variants of it, as well as a related red fluorescing protein (DsRED or RFP) from reef corals, are commercially available in the form of expressible plasmids. Functional transgenic expression of these fluorescent proteins is nearly universal in both eukaryotes and prokaryotes. Both the green and red fluorescing proteins have similar structural features, involving a beta-can fold structure enclosing a chromophore that is made via a reaction between 3 consecutive amino acids, serine, tyrosine and glycine. The quantum yield of fluorescence from the green fluorescent protein is near unity, while that from the red protein is apparently lower. Proteins with a variety of other wavelengths have also been characterized.

While certain kinds of screening procedures can be performed using fluorescence activated cell sorting (FACS), this does not allow for time dependent monitoring of individual cells or colonies. Most of the directed evolution studies performed to date have been performed with cells either in wells or on surfaces, using optical means to determine some activity over time often involving visual, qualitative screening of colonies on plates or in wells followed by manual selection of mutants that have enhanced activity in the protein of interest. Selection of cells may be based on a number of criteria, including color, morphology, size and fluorescence, depending on the protein of interest and the selectable marker chosen. When screening fluorescing cells, the process typically involves exciting cells with light and observing fluorescence from the genes or from molecules made by or associated with the genes in the cells. Visual screening is slow and not particularly amenable to automation. As a result, the number of cells that can be screened and selected for further processing is greatly limited.

Although electronic cameras have been used to record fluorescence levels from colonies of cells, only the total relative yield of the fluorescence at a particular wavelength is typically recorded. This does not distinguish between fluorescence amplitude, which depends on both the photophysical properties of the fluorophore and its concentration, and fluorescence lifetime, which depends only on the photophysical properties of the fluorophore. Thus, directed evolution procedures that rely on steady state measurements of fluorescence select for changes that can be in either the amount of or the chemical properties of the fluorophore, but cannot specifically select for changes in molecular properties independent of concentration.

Also, while the use of electronic cameras has made it possible to screen cells more rapidly, its application has been limited by the ability to manually select cells exhibiting desired traits. What is needed, therefore, is a more sensitive, higher resolution system that quantitates levels of fluorescence from a cell colony or from individual cells on a surface, thus allowing cell screening on the order of millions of cells per round of directed evolution, coupled with an automated system for selecting the colonies or cells of interest.

Thus, the ability to perform directed evolution using a high resolution fluorescent assay that is sensitive, amenable to automation, allows multiple readings of the same cell or colony on a surface over time and that distinguishes between fluorescence amplitude, fluorescence spectrum and fluorescence lifetime would be a significant asset for research as well as diagnostics and therapeutics.

### SUMMARY

Provided is a method for screening large numbers of individual cells or colonies based on fluorescence lifetime of fluorescent markers present in the cells. This can comprise providing a substrate with multiple locations, at least some of which contain one or more cells containing a fluorescent marker; directing a light source onto each location, thereby causing the fluorescent marker to emit fluorescent light, automatically detecting the fluorescent light, automatically measuring and recording an attribute of the fluorescent light and correlating the attribute of the fluorescent light with the location containing the cell with the fluorescent marker emitting the fluorescent light. The attribute can comprise at least one of fluorescence lifetime, intensity, spectrum, polarization and the like.

Also provided is a method for generating a high-resolution image map of cell fluorescence and using the image map to select cells exhibiting desired fluorescent properties.

Provided are methods for automatically selecting cells exhibiting an imagable property (indicating a desired characteristic of the cell), such as fluorescence, color, morphology, or any other property that may be detected and recorded, by applying a lethal light source to all cells in a sample that, if left in the dark, would result in the death of all the cells, then subsequently applying visible light to the cells exhibiting the imagable property (desired cells). In one embodiment, this comprises providing a substrate with multiple locations, at least some of which contain one or more cells expressing an imagable property wherein the cells have a photosensitive repair system; detecting and recording the imagable property; identifying and recording locations containing cells expressing the imagable property and locations not containing cells expressing the desired characteristic of the imagable property; exposing the substrate to lethal irradiation so as to kill a majority of the cells on substrate if not exposed to a repair light; and scanning a repair light (for example, visible light) through a high speed shutter and through an objective, wherein the shutter is open only when the objective is positioned over locations containing cells expressing the desired characteristic of the imagable property to thereby initiate DNA repair in the cells in such locations. The method can further comprise placing the sample in an environment that allows minimal or no exposure to repair light. Minimal exposure to repair light is exposure that is insufficient to initiate photosensitive repair.

In an alternative embodiment, provided is a method comprising providing a substrate with multiple locations, at least some of which contain one or more cells expressing an imagable property wherein the cells have a photosensitive repair system, detecting and recording the imagable property, identifying and recording locations containing cells expressing a desired characteristic of the imagable property and locations not containing cells expressing the desired characteristic of the imagable property, projecting lethal irradiation onto the substrate so as to kill a majority of the cells on substrate if not exposed to a repair light source, and projecting a repair light source (for example, visible light) only onto those locations containing cells expressing the desired characteristic of the imagable property to thereby initiate DNA repair in those cells.

Also provided is an apparatus for the automated screening and selection of cells based on fluorescence properties. This may be used with both prokaryotic and eukaryotic cells. This is useful in directed evolution methodologies but also may be used to screen and select cells in situ.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles:

FIG. 1 is a schematic diagram illustrating the general process for directed evolution;

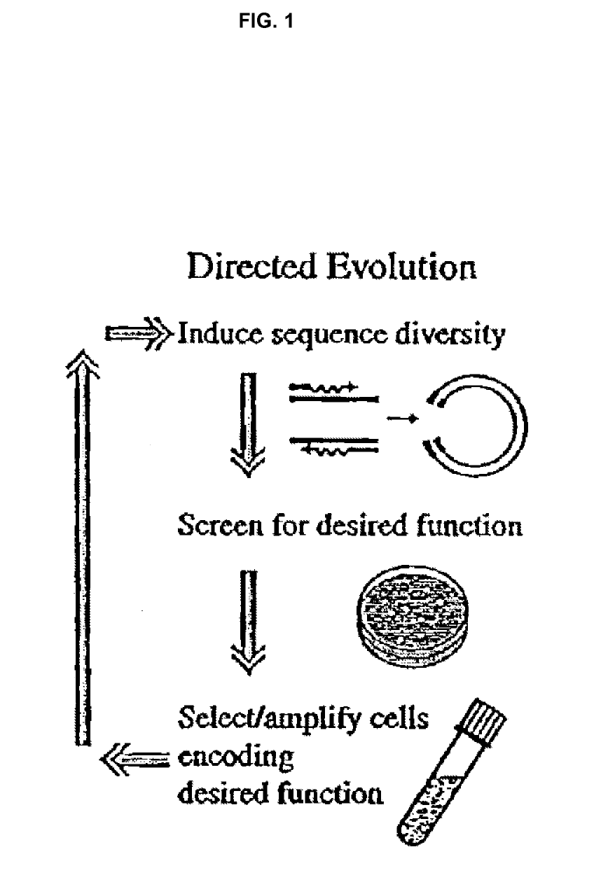

FIG. 2 is another schematic diagram illustrating the general process for directed evolution;

FIG. 3 is a schematic diagram of one embodiment of a fluorescence lifetime imaging system;

FIG. 4 is a schematic diagram of a four channel fluorescence lifetime imaging system;

FIG. 5 is an alternative embodiment of a fluorescence detection device;

FIG. 6 is a schematic diagram illustrating an example of the use of a visible light source, in this example a laser, to select cells based on their fluorescent properties;

FIG. 7 is a schematic diagram illustrating the use of a CCD camera to screen for fluorescence from cells and a digital light processor to specifically irradiate cells with ultraviolet light to kill individual cells that do not exhibit desired characteristics;

FIG. 8 is a flowchart illustrating methods provided; and

FIG. 9 is a flowchart illustrating methods provided.

### DETAILED DESCRIPTION

Before the present methods and systems are disclosed and described, it is to be understood that the methods and systems are not limited to specific synthetic methods, specific components, or to particular compositions, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The use herein of a term such as, “irradiating,” “scanning,” “exposing,” “projecting,” “illuminating,” and the like, does not specifically preclude the use of the other terms. For example, a method described using a scanning light source can also be implemented with a projection light source. The types of light sources disclosed herein can be used in any combination. For example, an excitation light source, a lethal light source, and a repair light source can all be a laser, a digital light processor, or any combination thereof.

“Optional” or “optionally” means that the subsequently described event or circumstance may or may not occur, and that the description includes instances where said event or circumstance occurs and instances where it does not.

The present methods and systems may be understood more readily by reference to the following detailed description of preferred embodiments and the Examples included therein and to the Figures and their previous and following description.

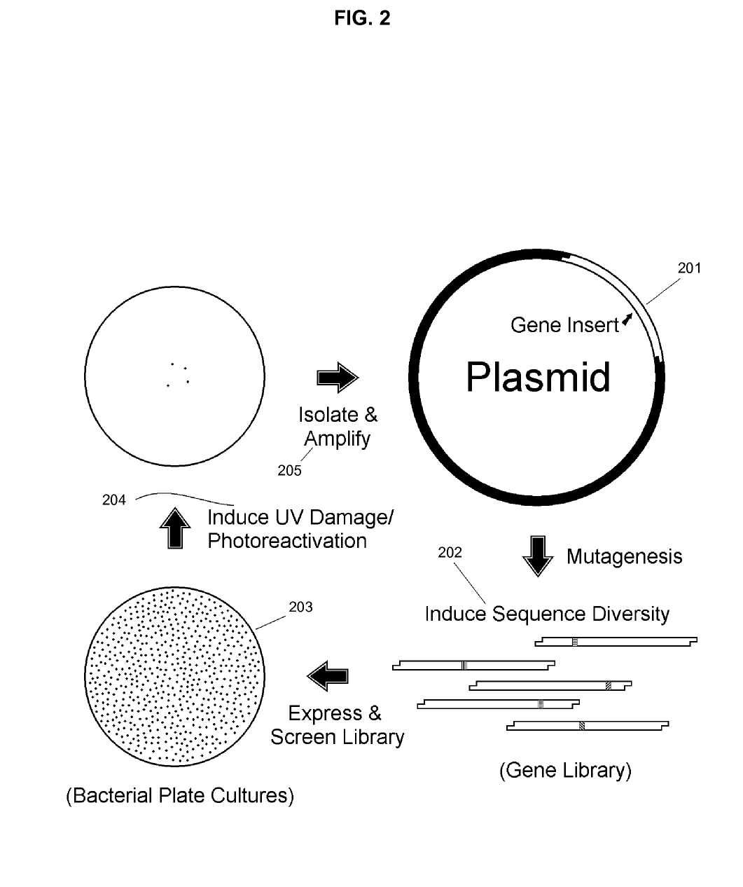

Provided are methods and systems that can be used as a means of positive selection (for example, in the process of directed evolution) by repairing cells with desired characteristics from libraries of mutants that have been deactivated via the induction of pyrimidine dimmers with UV light. An exemplary method is provided in FIG. 2. Starting at 201 with a plasmid containing a gene desired for evolution and study, mutations can be induced in the gene to create a library of mutants at 202. At 203, the library of mutants can be expressed in bacterial cell colonies on solid LB agar media and the colonies can be scanned with an excitation light source to generate an image map showing those colonies with desired functions. At 204 the agar plate can be exposed to a lethal light source, for example UV radiation, thereby inducing DNA lesions. Then desired colonies can be selectively exposed to a repair light source, for example light with a wavelength between 350-700 nm, to induce repair of the DNA lesions. The plate can be subsequently warmed in an incubator allowing only photoreactivated colonies to grow. At 205, the gene can be isolated and purified and the process can be repeated for further generations of directed evolution.

Also provided are means of high throughput selection of cells or colonies that can be completely computer controlled and allows for long-term monitoring of colonies before selection criteria are applied (not possible with techniques such as fluorescence activated cell sorting). This is critical for selection of cells that show differences between two conditions (for example, cells whose fluorescence level changes upon exposure to an analyte or over the course of an enzymatic reaction). The ability to perform positive selection greatly increases the selective advantage for the cells selected (negative optical selection is much more difficult to control). It can be also be performed orders of magnitude more rapidly than negative selection because only the desirable cells need to be specifically irradiated. This can be performed with any source of light of the appropriate wavelength including a scanning laser, a projector (LCD, DLP, and the like), a micromirror array based projection system, a physical mask and other approaches.

In one aspect, provided are methods and systems that allow for the positive selection of cells comprising light activated photorepair systems. The methods and systems exploit the innate ability of many types of both bacterial and eukaryotic cells to repair cyclobutylpyrimidine dimmers using the constitutive enzyme, DNA photolyase. This selection can be performed by determining first which cells or colonies of cells out of a large number of cells or colonies one wishes to keep and which one wishes to kill. This can be performed by scanning an excitation light source (such as a laser, a lamp, a light emitting diode and the like) across the colonies and looking for particular fluorescence or absorbance or morphology characteristics (there are many ways to characterize cells and colonies, optical methods are only one approach). A majority (or all) of the cells are then irradiated with lethal (such as ultraviolet light from a laser, a lamp, a light emitting diode and the like) light at a level that would kill the vast majority of them if not exposed to a repair light. The method can further comprise placing the sample in an environment that allows minimal or no exposure to repair light. Minimal exposure to repair light is exposure that is insufficient to initiate photosensitive repair. Subsequent application of a repair light source (such as visible light from a laser, a lamp, a light emitting diode and the like) only to the cells or colonies one wishes to save results in repair of the damage to the DNA caused by the UV light. The cells irradiated by visible light are much more likely (about 10,000 fold more likely in most cases) to survive than the cells not exposed to a repair light.

The methods and systems provided allow for screening large numbers of individual cells or colonies of cells using scanning microscopy coupled with fluorescence lifetime, fluorescence spectra and/or fluorescence polarization measurements and analysis, using time-correlated single photon counting or other approaches such as streak camera measurements. Both the imaging of the fluorescence lifetime data from cells and/or colonies on a surface and the analysis of this data can be controlled and performed in an automated and rapid manner using a computer. This screening method can then be used with either light-mediated patterned cell growth methodologies, as further provided herein, or mechanical methods to select individual cells or colonies based on their fluorescent properties.

The methods and systems provided are improvements over current methods for screening cells. Automated scanning of the fluorescent properties of cells or colonies enables a large number of colonies to be screened rapidly and automatically. In the practice of the methods and systems provided, a slide containing millions of cells can be examined in minutes. Also, the method allows one to determine independently the lifetime, intensity, polarization, and the spectrum of the fluorescence. Current methods of screening involve either a manual or automated survey of total fluorescence, which depends on both the lifetime and the amplitude of the fluorescence. By distinguishing between lifetime and amplitude, one can determine whether changes in fluorescence are due to changes in numbers of fluorophores or changes in the excited state lifetime (i.e., the chemical properties) of the fluorophores. In addition, the determination of spectral properties can make the measurements sensitive to structural or environmental changes. Finally, the measurement of polarity can be used to determine how rapidly fluorophores are moving (and whether that changes due to, for example, interaction with other molecules), how easily they transfer excited state energy to neighboring molecules (for example when two fluorescence tags interact in a cell), or the relative orientation of static molecules in the sample.

The methods and systems provided allow for automated selecting of cells that exhibit desired characteristics. In one embodiment, this method utilizes a lethal light source such as a laser a light emitting diode or a lamp to illuminate cells immobilized on a surface. The cells are of a type that contain light activated photorepair systems (either naturally or engineered). The cells can be any type of living cell. The lethal light source can be, for example, any lamp, light emitting diode or laser that emits light and that has an intensity at the surface capable of killing a majority (or all) of the cells by introducing damage that can be repaired by subsequent exposure to a repair light source, for example, visible light. For example, the cells can be bacterial cells and the lethal light source can be a bacteriocidal UV light source. The target cells that are exposed to the lethal light source in this way would be killed if not exposed to a repair light source after exposure to the lethal light source. After the lethal light source is turned off, subsequent application of a repair light source only to the cells or colonies desired to survive results in repair of the damage to the DNA of those target cells caused by the lethal light source. This method can be performed with a repair light source of patterned visible light of the appropriate wavelength (for example, between and including 350-700 nm, including a scanning laser (or focused lamp or light emitting diode), a projector (LCD, DLP, and the like), a micro-mirror array based projection system, and the like. Patterning can be accomplished by any means known in the art, including but not limited to a physical mask, selective projection, and the like.

In an alternative embodiment of this patterned growth cell selection method, a computer-controlled projection device, such as a micro-mirror array or a liquid crystal display system, can be used to project an image onto the cells after application of the lethal light source. Cells onto which this image is projected initiate DNA repair, resulting in a patterned growth of cells. As used herein, projection can be accomplished by directing a specific image onto a substrate, by providing a mask to thereby cover portions of the substrate not to be irradiated, or by other methods known in the art.

By employing the present methods and systems, cells can be selected with high spatial resolution, and large numbers of cells can be processed. Importantly, this cell selection can be done strictly based on function, as manifested in some detectable property of the cell such as fluorescence or absorbance. This is in contrast with other high throughput selection procedures that utilize large numbers of cells, but require that the selected trait confer a significant growth advantage. This process can be coupled with high throughput imaging of cell fluorescence using either a sensitive charge couple device based camera (CCD) camera or a scanning microscope.

The methods and systems permit selection of desirable cells in directed evolution techniques, since cells can be selected with great resolution at sub-visual sizes, allowing a vast number of cells to be processed at once, without the need for antibiotic resistance markers or growth on selective media lacking required nutrients. The methods and systems can also be used in color-based assays for transformation of bacterial cells with plasmid DNA, obviating the need for antibiotic resistance. Further, cell patterning can be used with essentially any cell type, including yeast and many mammalian cells.

Spatially Imaged Fluorescence Detection Devices

The spatially imaged fluorescence lifetime detection device comprises a scanning microscope system with a nanopositioning or micropositioning stage, or a laser scanning system, modified by the inclusion of a pulsed excitation light source, a photon counting detector and appropriate time correlation electronics. In one embodiment, a confocal microscope is used, although other microscope systems may also be used. The positioning capability can be in either two or three dimensions, and allows computer controlled movement system that can position the focal point of a beam on a sample with submicron accuracy. Such positioning stages or scanning systems are commercially available from, for example, Mad City Labs (Madison, Wis.; Nanoh100-xy), PI (Physics Instruments, Germany) or Brimrose Corporation of America (Baltimore, Md.). Alternatively, the stage may be kept stationary while the beam is moved relative to the stage.

The pulsed excitation light source can be any laser or other light source with a high repetition rate and a short pulse width, generating pulses at greater than 10 Hz. In one embodiment, an actively mode-locked NdYAG laser is used, generating pulses at 80 MHz, which, after compression, are 5 ps in duration. The wavelength used to excite the sample varies according to the sample. In another specific embodiment, an ultrafast titanium sapphire oscillator is used, pumped by a continuous laser source such as a diode-pumped NdYAG laser. The oscillator produces pulses of about 100 femtosecond duration at a repetition rate of 80 MHz.

In one embodiment, the fluorescence lifetime measurement may be performed by time correlated single photon counting. This involves using a photon counting device comprising any detector capable of detecting and counting photons, generating electrical pulses for each photon detected. In one embodiment, an avalanche photodiode is used. Alternatively, a photomultiplier tube is employed. Such devices are well known in the art.

The time correlation electronics is any device that can receive information both from the photon counting device and from the laser, or from a fast photodiode associated with the laser, and record time in two dimensions. Preferably, the device uses time correlated single photon counting (TCSPC) to determine the time between a laser pulse and the resulting photon emission (i.e., the excited state lifetime of the molecule giving rise to the photon, generally in the nanoseconds time frame) and it records the time at which the photon arrives, in the lab time frame, typically with microsecond to millisecond accuracy. Such time correlation electronics are commercially available from, for example, Becker & Hickl (Berlin, Germany) or PicoQuant (Berlin, Germany).

In another embodiment, the detection of desirable cells can comprise a streak camera system (a device for measuring fluorescence as a function of time with picosecond or subpicosecond resolution). Such systems are made by, for example, Hammatsu Corp. This device can be used directly with a microscope in much the same way as the time correlated single photon counting system described above. Such a device can also be configured to provide detailed spectral information about the fluorescence.

In the practice of the device, a beam from the high repetition rate pulsed laser is passed into the microscope, reflected from a dichroic mirror, and used to excite a sample. Preferably, the sample sits on a 3-D translation/positioning stage or the laser position is controlled by a scanning device such as a rotating mirror or an acousto-optic scanner (these devices will be collectively referred to as “positioners”) and its position relative to the focused laser beam is controlled by the computer, thus allowing scanning of the sample. The sample can be comprised of single cells or colonies of cells sitting on, or embedded in, a solid substrate so that their positions do not vary over the period of time required to obtain the image. The cells may be either prokaryotic or eukaryotic, with at least some portion of the cells exhibiting fluorescence, or other imagable property, when excited.

Upon excitation, the sample emits a fluorescent signal that passes through various optical elements. In one embodiment, the fluorescence passes through the dichroic mirror, as the fluorescence is at a wavelength that is not reflected by the dichroic mirror. Each photon emitted by the sample is counted at the detector and the time of arrival of each emitted photon relative to the laser pulse is correlated, stored and analyzed on the computer.

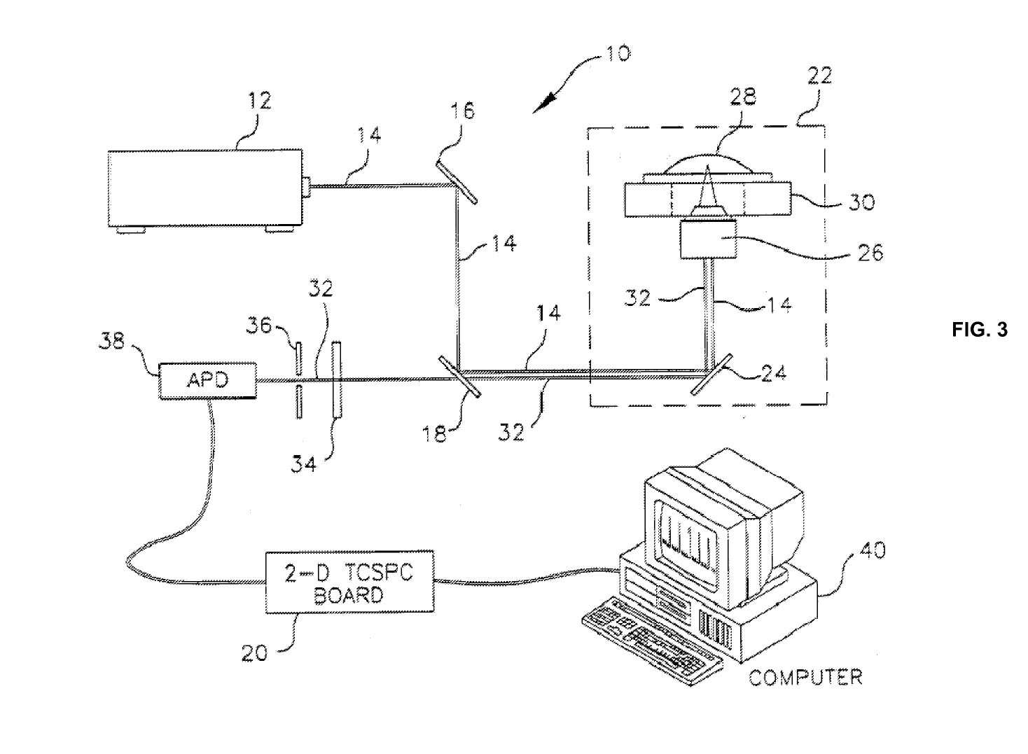

FIG. 3 shows one embodiment of the spatially imaged fluorescence lifetime detection system 10. A high frequency (greater than 10 Hz) laser system 12 is used as the excitation light source. The laser emits a light beam 14, which is directed via the use of a mirror 16 to a dichroic mirror 18. The laser is connected to a 2-D TCSPC board 20, which receives an input from the laser that marks the time at which the laser pulse was initiated.

The dichroic mirror 18 reflects the laser light beam 14 into the microscope system 22, where it is directed via additional mirror(s) 24 to the objective lens 26. This lens system focuses the beam onto the sample 28. The sample is attached to a computer-controlled positioning stage 30.

The laser beam excites molecules within at least some of the cells on the stage, causing them to emit light as fluorescence. Some of this fluorescence 32 is captured by the objective lens 26 and passed back into the microscope along the same path through which the laser light beam 14 entered. The fluorescence is reflected from mirror 24 to the dichroic mirror 18, where the fluorescent light passes through, as the dichroic mirror is selected to reflect light at the wavelength of the laser light beam but transmit light at the wavelength of the fluorescent light. The fluorescent light 32 then passes through a filter 34 to remove any remaining laser light while efficiently passing light in the wavelength region of the fluorescence and, optionally, through a confocal pinhole 36 (typically on the order of 50 to 150 microns in diameter and translatable along the axis of the laser beam) to better define the volume of sample being probed.

The fluorescent light is detected by an avalanche photodiode 38, which generates electrical pulses for each photon of fluorescent light it detects. These pulses are transmitted to the TCSPC board 20. The TCSPC board records the time at which the photon arrived and uses time correlated single photon counting to determine the time between the laser pulse and the photon emission. This information is transmitted to a computer 40, where it is stored and analyzed. Optionally, computer 40 is interfaced with the positioning stage 30.

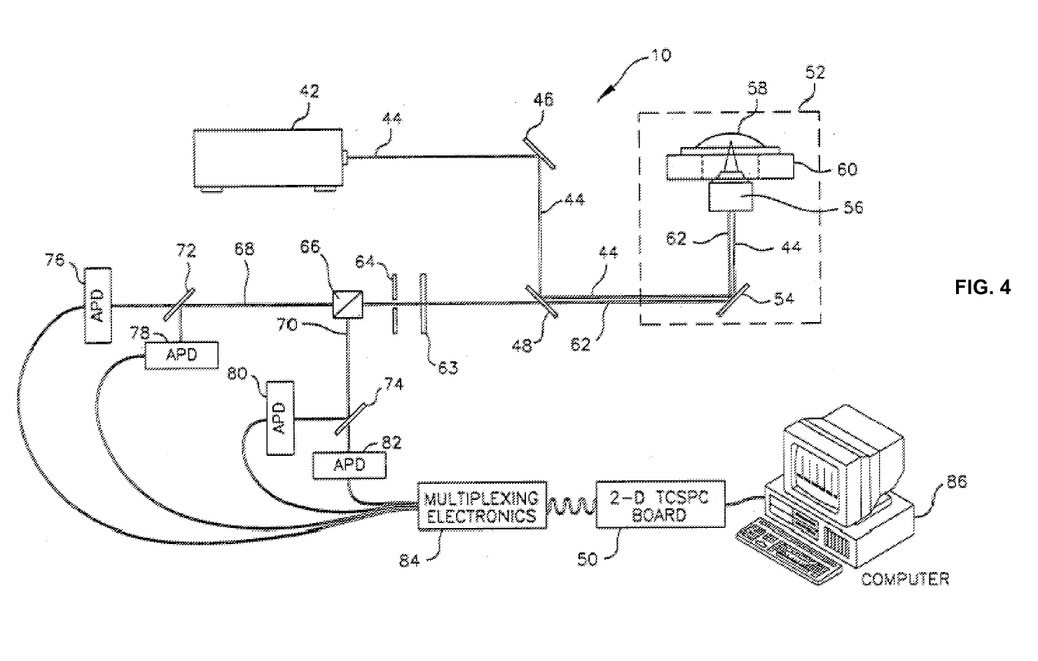

An alternative embodiment of a fluorescence detection device is shown in FIG. 4. In this embodiment, a four channel system records not only the excited state lifetime of each fluorophore that gives rise to each photon detected, but also records the polarization of the photon and wavelength region in which it was emitted. This additional information can also be used to determine which cells or colonies exhibit the most desirable characteristics; for example, those cells containing the most desirable gene products in the directed evolution process.

In this embodiment, a laser 42 emits a light beam 44, which is directed via the use of a mirror 46 to a dichroic mirror 48. The laser is connected to a 2-D TCSPC board 50, which receives an input from the laser that marks the time at which the laser pulse was initiated.

The dichroic mirror 48 reflects the laser light beam 44 into the microscope system 52, where it is directed via additional mirror(s) 54 to the objective lens 56. This lens system focuses the light beam onto the sample 58. The sample is attached to a 2-D positioning stage 60 controlled by a computer (not shown).

As the laser beam excites molecules within at least some of the cells on the stage, fluorescent light 62 is emitted, some of which is captured by the objective lens 56 and passed back into the microscope along the same path through which the laser light beam 44 entered. Upon reaching the dichroic mirror 48, the fluorescent light passes through the dichroic mirror, through a filter 63 and, optionally, through a confocal pinhole 64.

The fluorescent light then enters a polarizer 66, emerging from the polarizer in two perpendicular planes 68, 70 as polarized light, each of which enters a wavelength separator 72, 74. Polarized light passing through the wavelength separator is again split into two paths of light, each of which is detected by an avalanche photodiode 76, 78, 80, 82. The avalanche photodiode generates electrical pulses for each photon of fluorescent light it detects. These pulses are transmitted through multiplexing electronics 84 to the TCSPC board 50. The multiplexing electronics comprise a circuit which adds a different period of delay time to the pulses arriving from different channels (Becker & Hickl, Berlin, Germany). In this way the TCSPC board is able to differentiate between the signals from the four different detectors. The TCSPC board records the wavelength region and polarization of each photon, in addition to the lifetime of the excited state that gave rise to the photon. These attributes can all be recorded along with the arrival time of each photon in the lab time frame with a millisecond resolution. This information is transmitted to a computer 86, where it is stored and analyzed.

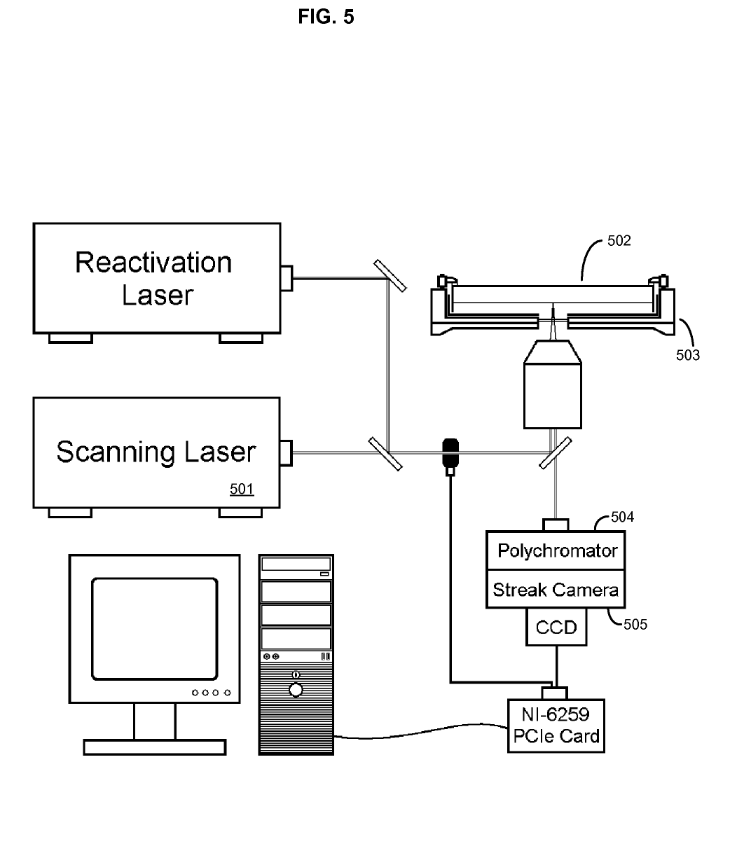

Another alternative embodiment of a fluorescence detection device is shown in FIG. 5. Colonies on agar plates may also be imaged using a streak camera imaging system as illustrated in FIG. 5. The streak camera imaging system can generate a repair image that can be used as the basis for cell selection. The streak camera imaging system makes use of a scanning laser 501 as an excitation light source to excite fluorophores in the colonies in the sample 502. The sample is attached to a positioning stage 503 controlled by a computer (not shown). The resulting emission photons from each colony (or sample point on the plate) are collected and sent through a spectrophotometer, polychromator 504. The polychromator 504 can select a specific wavelength region of the emission. This wavelength region can be located anywhere in the UV, visible or near infrared spectrum and can have a spectral width which is variable depending on the gratings used in the polychromator 504. For example, gratings can allow spectral widths of 100 nm, 160 nm or 320 nm. This emission is then directed to a streak camera 505. The entire emission spectrum can be recorded as function of time. In this way it is possible to determine the fluorescence intensity as a function of both wavelength and time for each colony with temporal resolution of a few picosconds and spectral resolution of less than 1 nm.

Various other methods for imaging cells can also be used. For example, a charge couple device based camera (CCD camera) may be used. It is also possible to monitor absorbance in a spatially resolved fashion or to use a scanning probe microscope to generate an image of the morphology, electrical characteristics, surface properties, etc., of cells. Any imaging system with sufficient spatial resolution to resolve the features important in identifying cells with desired properties may be employed.

Light Mediated Patterning in Cell Selection

A lifetime image can be determined by correlating the excited state lifetime measured by the spatially imaged fluorescence lifetime detection system with the position of the positioner at the time of the measurement within the lab. This time frame can be used to determine which of the cells or colonies in the sample have the desired characteristics. Then, any of several computer-controlled methods for rapidly selecting individual cells or colonies can be employed. For example, any of several automated mechanical methods for picking cell colonies and moving them to a clean substrate can be used. Whatever selection method is used, the lifetime image of the cells or colonies is stored on a computer and the computer can then be used to automatically decide which cells should be selected, using this information to initiate an automated procedure for cell selection.

Provided are methods for selecting cells based on patterned cell growth. This method employs a positive selection strategy, in which a majority of cells (or all the cells) are exposed to a lethal light source, sufficient to kill most or all of the cells if not exposed to a repair light source, and subsequently applying a repair light source, sufficient to initiate DNA repair, to those cells identified to exhibit the desired characteristic. Alternatively, a repair light source in the form of a repair “image” can be projected onto the sample that initiates repair in the desirable cells.

In one embodiment, fluorescence from cells on a surface is recorded by a scanning fluorescence microscope capable of recording both the fluorescence amplitude and its lifetime, via the use of single photon counting technology, as described above. The image thus obtained of the fluorescence on the surface is used to determine which cells or colonies exhibit the desired fluorescence characteristics. This information is processed and a new image (the “repair image”) is generated by the computer. A lethal light source can be applied to the cells on the surface, which would ultimately lead to cell death if the cells are not exposed to a repair light source. The cells can then be subjected to projection of the repair image from the repair light source.

The repair image is designed to indicate where to apply visible light so as to only irradiate the desirable cells (that is, those exhibiting the desired fluorescence characteristics) under conditions that allow for DNA repair in those cells, leaving the cells with undesirable fluorescence properties to die. For example, the repair image can be projected by scanning a visible light source with a wavelength in the range of about 350 nm to about 700 nm across the surface of the plate. This can be performed with any source of patterned light of the appropriate wavelength including a scanning laser, a projector (LCD, DLP, and the like), a micro-mirror array based projection system, a physical mask and the like.

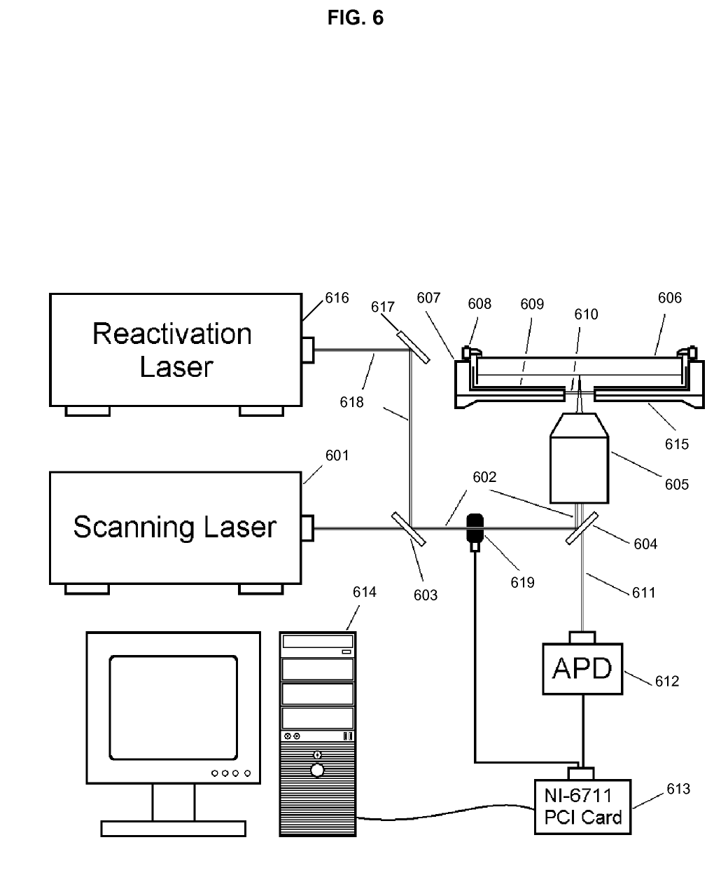

FIG. 6 shows an example of the use of a repair light source, in this example a laser, to select cells based on their fluorescent properties. In FIG. 6, a scanning laser system 601 is used as an excitation light source. The laser emits a light beam 602, which is directed to a dichroic mirror 603.

The light beam 602 is reflected into a microscope system by dichroic mirror 604, into the microscope objective 605. The microscope objective 605 focuses the light beam onto a sample of cells located on the surface of a plate 606. The cells can be, for example, bacterial cells that were grown on solid LB agar media, expressing mutants of a target protein. The plate 606 is held securely in a kinetic Petri plate holder 607. Spring-loaded clamps 608 press the Petri dish down against a Petri plate gasket 609 to create an air-tight seal. The light beam 602 passes through a quartz window 610 in the base of the kinetic plate holder 607, exciting a target protein or a reporter protein for the target, causing some portion of the cells to emit fluorescent light 611.

Some of this fluorescence is captured by the microscope objective 605 and passed back into the microscope along the same path through which the light beam 602 entered. Upon reaching the dichroic mirror 604, the fluorescence passes through as the dichroic mirror is designed to reflect light at the wavelength of the laser but transmit light at the wavelength of the fluorescence.

The cell fluorescence is collected and counted by an Avalanche Photodiode (APD) 612. A National Instruments 6711 PCI card 613 collects the data from the APD. Computer 614 records the data from the NI-6711 card and provides control of scan motion. The computer 614 can record a property of the fluorescence, for example, at least one of the intensity of the emission, the lifetime of the emission, the polarization of the emission, and the spectrum of the emission. The microscope stage 615, under the control of computer 614, can scan the plate to generate time resolved images of the fluorescent bacterial colonies on the plate. The images thus generated are stored and analyzed in a computer 614 and used to determine which cells or colonies on the surface should receive repair irradiation from a repair light source, such as reactivation laser 616. After scanning, the kinetic plate holder 607 is detached and the colonies are exposed to a lethal light source, such as UV radiation (not shown). The kinetic plate holder 607 is then returned to the stage and the reactivation laser 616 is used to target individual colonies for photoreactivation. Mirror 617 and dichroic mirror 603 are used to direct the reactivation light beam 618 along the same pathway as the scanning laser. The NI-6711 PCI card 613 provides a signal such as, for example, about a 5V signal to control a shutter 619 for the reactivation laser 616 to ensure reactivation light 618 is only directed toward the colonies selected for reactivation. Plates can then be incubated at about 37° C. overnight, resulting in only reactivated colonies growing to a working size.

It is also possible to use an ultrafast laser pulse (on the order of a few hundred femtosecond duration) in the near infrared as the excitation light source for performing both the measurements of excited state lifetimes and for positive cell selection using imaged light. This is involves multiphoton excitation of either the fluorophore being used to probe function or the chromophore in the DNA repair enzyme (DNA photolyase) that results in DNA repair. The very high peak intensity of short pulses make it possible for the fluorophore, such as GFP, to absorb two photons of near infrared light and then to fluoresce in the usual visible region. Similarly, the chromophore in the light-dependent DNA repair enzyme can absorb two photons and perform its function just as it would with a single photon in the blue region of the visible spectrum. Using an ultrafast laser pulse in the near infrared in this manner has several advantages. First, since the excitation is in the near infrared, it is very well separated spectrally from the fluorescence. This decreases the background due to scattering of various kinds as well as other fluorescent materials. Second, because the process depends on multiple photons, the volume of material where the photon density is high enough to cause the multiphoton absorption is smaller, increasing the spatial resolution of the technique. Finally, by using near infrared or even infrared light as the source of photons for the multiphoton excitation, it is possible to excite fluorophores or chromophores in cells below the surface of a sample allowing three dimensional mapping of the fluorescence and cell selection. This deep probing occurs without exciting fluorophores or chromophores in the cells above, because the intensity of light will only be great enough at the focal point of the beam to perform the multiphoton excitation.

The intensity and wavelength of the laser beam used for multiphoton excitation screening and cell selection depends both on the specific fluorophores or chromophores being used and on the geometric constraints of the sample. The wavelength used for screening needs to be a multiple of the absorbance wavelength preferred for the fluorophore or chromophore to be excited. The power level of the laser is also dependent on the nature of the fluorophore or chromophore, the concentration of the fluorophore or chromophore and the size of the region to be excited at any given time. Generally, the minimum laser intensity required to obtain a substantial signal from the fluorophore, or function from the chromophore, should be used, and this can be determined by performing test scans with increasing light levels. Multiphoton excitation of DNA and/or protein molecules in the cell are possible by picking an excitation wavelength that is a multiple of a wavelength in the absorbance range of these molecules (about 190-290 nm). The intensity required will depend on the multiphoton absorption cross section and the cell type. Again this can be determined empirically by increasing the intensity in a test case until high resolution cell imaging and selection is achieved. Multiphoton beams have previously been used for “nanosurgery” at the subcellular level.

In an alternative method of light mediated patterning in cell selection, fluorescence from cells on a surface is recorded by a CCD camera. The image of the fluorescence on the surface is used to determine which cells or colonies contain the desired fluorescence characteristics (i.e., which cells are expressing biomolecules that have the desired traits). This information is processed and a repair image is generated by the computer. The repair image is designed to expose the cells to visible light that have desirable fluorescence under conditions which will allow those cells to initiate DNA repair and leave the cells with undesirable fluorescence to die. Alternatively, the repair image can be designed to expose visible light to all cells with fluorescent activity above a predetermined threshold. The surface is then exposed to a lethal light source (for example, with a lamp or laser system).

The repair image can then be projected using a repair light source (for example, a lamp or a laser system) in conjunction with a microscope or a computer-controlled imaging system such as micro-mirror array chips (digital light processors or DLPs) or liquid crystal projection units that are commonly used for projecting computer generated images on screens (available from Texas Instruments, Dallas, or InFocus Corporation, Wilsonville, Oreg.). The repair light source (lamp or laser) is selected to emit light at a wavelength or range of wavelengths suitable for initiating DNA repair in selected cells. Also, the imaging optics are selected both to be appropriate for the size of the image to be generated and for the wavelength region of light used. Finally, appropriate filters can be used to select the desired wavelength regions of light. In particular, a high quality lens system with low optical aberrations is used such that the inherent resolution of the instrument is maintained when the image is reduced to the size of the target.

In one embodiment, the computer storing the repair image is connected to an InFocus® model projector, which uses the video output from a computer to display an image onto a screen (InFocus Corporation, Wilsonville, Oreg.). The focal optics of the projector are replaced by a 50 mm Nikon® lens area, so that the output can be focused on the cells with image features having the proper size and alignment (Nikon, Inc., New York, N.Y.). Other lens systems can also be used, depending on the size of the target area. The projector uses an aluminum micro-mirror array that is electronically controlled. Suitable chip dimensions are 1024×786 pixels, although other dimensions may be used depending on the desired resolution. When focused on an agar plate containing cells, the image size is approximately 11 cm by 8.5 cm, producing a pixel size of approximately 0.07 mm/pixel. Such an optical arrangement allows selective imaging and repair initiation in a library of, for example, colonies containing several hundred thousand members.

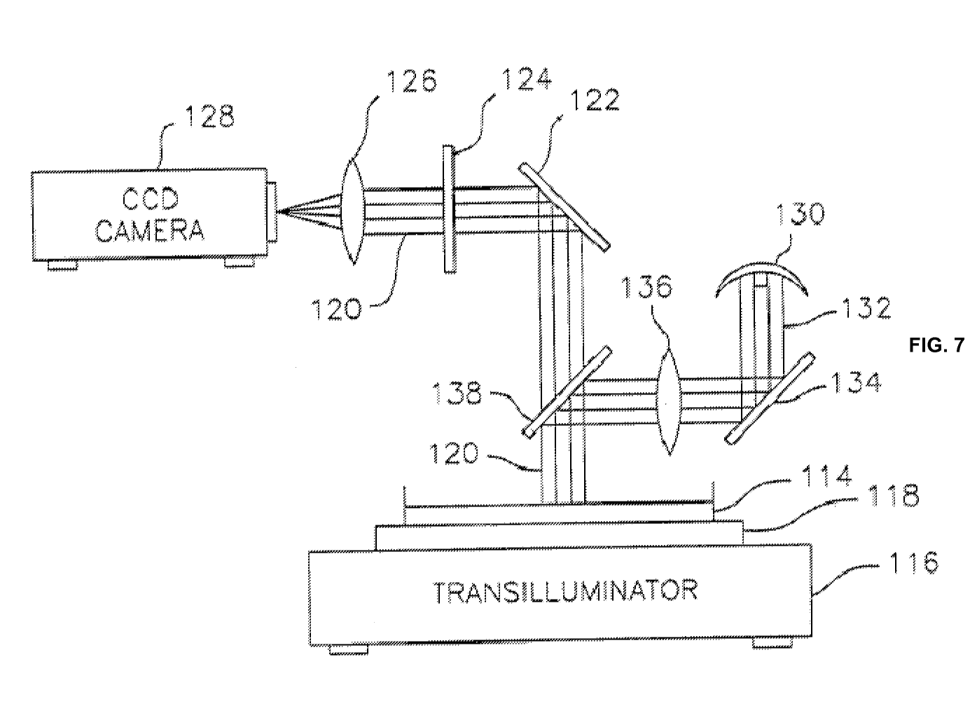

FIG. 7 shows an embodiment of a CCD camera being used to image the fluorescence from cells. This information is then used by the computer to generate the repair image to be projected onto the plate of bacteria after application of a lethal (i.e., bacteriocidal) light source to a majority (or all) of the cells on the plate. In this embodiment, the cells to be screened and selected are grown in a plate 114 on agar or other solid substrate, preferably which supports growth of the cells. The plate is supported on a transilluminator 116, with a filter 118 between the plate and the transilluminator. The filter 118 is selected to permit a wavelength or wavelengths of light suitable for exciting fluorescence of the cells to pass from the transilluminator 116 to the cells on the plate 114, but not wavelengths that would interfere with the detection of fluorescence from the cells.

Fluorescence 120 emitted from the excited cells is reflected from a mirror 122, through a filter 124 and a lens 126 into a CCD camera 128. The fluorescence image detected by the CCD camera is stored in a computer (not shown) and is used to generate the repair image designed to selectively initiate DNA repair in cells exhibiting the desired traits. An ultraviolet light source (not shown) emits UV light over the entire surface containing the cells, killing some portion (or all) of the cells.

Subsequently, a repair light source 130 emits visible light 132 into a digital light processor 134, which projects the visible light image through a lens 136 and onto a dichroic mirror 138, selected to allow the fluorescence used for imaging by the CCD camera 128 to pass through and visible light to be reflected. The dichroic mirror 138 reflects the visible light image onto the cells in the plate 114, selectively initiating DNA repair in some portion of the cells.

Various other methods for imaging cells can also be used. Alternatively, one can use a scanning fluorescence microscope (one example is a scanning microscope capable of determining for example, the lifetime, intensity, polarization, and the spectrum of the fluorescence). It is also possible to monitor absorbance in a spatially resolved fashion or to use a scanning probe microscope to generate an image of the morphology, electrical characteristics, surface properties, etc. of cells. Any imaging system that works with high enough spatial resolution to resolve the features important in determining which cells have the most advantageous properties for the directed evolution project of interest can be used.

Use of Computer Interfaced Scanning Fluorescence Microscope in Cell Screening and Selection in Directed Evolution Methodologies

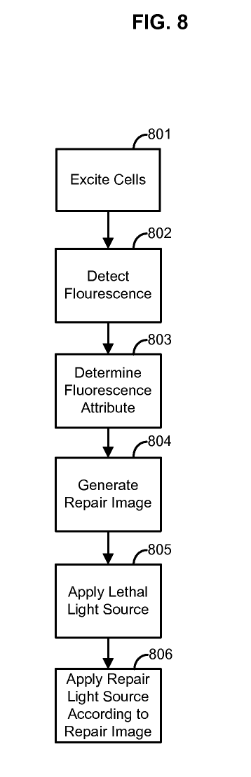

As is apparent from the description above, a computer interfaced scanning fluorescence microscope is useful in directed evolution methodologies where fluorescence, or other imagable property, is used as an indicator of protein function to screen and select cells for desired functional characteristics. An overview of this method is shown in FIG. 8.

A sample of the cells to be screened is placed on a positioning stage and positioned under the objective of the computer-interfaced scanning microscope. The computer moves the positioning stage, recording the position of the stage relative to the objective in lab frame. At the same time, the cells are excited by an excitation light source at 801, such as a mode-locked laser (though non-pulsed sources of light can also be used if fluorescence lifetime is not a desired parameter), through the objective of the scanning microscope. Some portion of the resulting cell fluorescence passes through the objective and is directed to an avalanche photodiode or photomultiplier tube or a streak camera system for detection at 802, subsequently, appropriate electronics (streak camera controller or a TCSPC computer card) can be used to process the information and transfer it to a computer, where an attribute of fluorescence can be measured and recorded at 803, such as the fluorescence lifetime, intensity, spectral and polarization information.

The fluorescence data is correlated with the position of the positioning stage (and thus the position of the sample), generating a high-resolution repair image map of individual cell (or colony) positions based on at least one of lifetime, intensity, polarization, and the spectrum of the fluorescence at 804.

This high-resolution map image can be stored as a repair image for use in identifying individual cells or colonies expressing desirable functional traits, as measured by at least one of lifetime, intensity, polarization, and the spectrum of the fluorescence. These cells or colonies are then selected for future rounds of directed evolution.

A lethal light source, such as a bacteriocidal light source, can then be applied to all the cells, such that the cells will ultimately die if not exposed to a repair light source at 805. Then, using the repair image map, a repair light source is applied to the desirable cells at 806 to initiate photosensitive DNA repair. The repair light source can be a visible light source directed through a fast shutter and into the objective of the scanning microscope, where it is scanned across the sample of cells. Both the shutter and the nanopositioning stage can be controlled by the computer such that the shutter is open when desirable cells or colonies are positioned under the objective, initiating DNA repair in those cells, while the shutter is closed when undesirable cells or colonies are positioned under the objective.

As is apparent, this method selectively and automatically initiates DNA repair in desirable cells (or colonies) one cell (or colony) at a time, greatly increasing the number of cells which can be screened and selected during directed evolution.

Use of Light Mediated Patterning Using Imaging of Repair Light in Directed Evolution Methodologies

Provided herein is another overview of the use of light mediated patterning using imaging of repair light in directed evolution methodologies where fluorescence, or other imagable characteristic, is used as an indicator of protein function, cell morphology or activity of cellular components. In this method, a sample is subjected to lethal UV irradiation which would result in cell death if not exposed to a repair light source, and a high resolution repair map is projected onto the sample, selectively initiating DNA repair in populations of desirable cells while permitting undesirable cells to die.

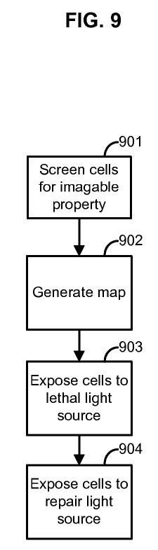

As shown in FIG. 9, a sample of cells to be screened for an imagable property, such as fluorescence, is excited by a light source such as a transilluminator, causing some portion of the cells to fluoresce at 901. This fluorescence is detected and recorded by an electronic camera, such as a CCD camera, interfaced with a computer. The fluorescence data is used to create a high-resolution map correlating fluorescence with cell (or colony) position within the sample.

The fluorescence image is used to generate a high-resolution repair map of cells (or colonies) expressing desirable functional characteristics at 902. A lethal light source, such as UV light, is projected onto the sample in order to kill all the cells if not exposed to a repair light source at 903. A repair light source, such as visible light, is projected onto the sample in the form of the repair image in order to initiate DNA repair in desirable cells at 904. The projection of the repair image may be controlled by a digital light processor interfaced with the computer storing the repair image.

Because the repair image is a high resolution map based on fluorescence data correlated to desirable functional characteristics, this method of light mediated patterning is a rapid and efficient way to simultaneously select large numbers of cells or colonies for further directed evolution studies.

As is apparent, the detection technique used to generate the high resolution map may by varied depending the cells desired. So long as the property of interest, whether it is cell morphology, calorimetric reactions or the like, can be imaged, a high resolution map can be generated for use in patterned cell selection as described for fluorescing cells.

The cell screening and selection methods described above are not limited to use with bacterial cells. The methodologies provided have applications for eukaryotic cells as well. For example, patterned cell selection can be used for the selection of yeast cells, which are often used in various techniques in which libraries of gene sequences are generated and specific colonies are selected. Patterned cell selection can also be used in the selection of mammalian cells for similar reasons.

### EXAMPLES

The following example is put forth so as to provide those of ordinary skill in the art with a complete disclosure and description of how the compounds, compositions, articles, devices and/or methods claimed herein are made and evaluated, and are intended to be purely exemplary and are not intended to limit the scope of what is claimed. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

E. coli cells were electroporated with a plasmid that has had random mutations introduced into its primary protein-coding sequence, via error-prone PCR, creating a library. For this example, the protein was a fluorescent protein. The cells were spread on LB medium Petri plates and allowed to grow for 8 hours at 37° C. at an approximate density of 3,000 cells per cm². The colonies that formed were then scanned by a laser to determine which cells were expressing the fluorescent protein with the most desired characteristics. The plates were next exposed to light emitted from a UV lamp (200-300 nm) for 3 minutes. This formed pyrimidine dimers that would result in cell death if they were not repaired. The dimers may be repaired if DNA photolyase binds to the dimer and absorbs light at approximately 370 to 410 nm. This process is known as photreactivation. The colonies that were expressing fluorescence with the desired characteristics were exposed to photoreactivating light by specifically pointing the laser at these colonies and not their neighbors. Irradiated cells were effectively reactivated while the cells that were not exposed to the photoreactivating light died. Surviving cells were scraped off the plate, grown in liquid culture and their plasmids recovered, subjected again to error prone PCR and various additional rounds of mutagenesis and selection were performed to develop a protein with optimized characteristics. This approach can be used for all cells that utilize a light activated DNA-repair process. Two photon irradiation can be used for the reactivation of cells or colonies in order to increase resolution and contrast and thus increase the selective advantage of the targeted cells.

### Example 2

Provided is a protocol describing an experiment in which fluorescence bacterial colonies are selected using the methods and systems described herein from a large background of nonfluorescent colonies.

On day one, DH5α E. coli cells (a common strain of commercially available E. coli) were plated (distributed and allowed to grow) on LB agar plates (LB broth, 1.5% Agar, 100 μg/mL Ampicillin). On one of the Petri dishes used, the cells contain the plasmid, pGFPuv (a plasmid that confers fluorescence by expressing green fluorescent protein). On a second dish, cells containing a similar plasmid that does not confer fluorescence (the GFPuv gene has been replaced with λ-phage DNA) are plated. The nonfluorescent cells are called Dummy#4 cells.

On day two, overnight liquid cultures of both pGFPuv and Dummy#4 cells (5 mL LB broth, 10 μL of 50 mg/mL Ampicillin) were innoculated. The cultures were grown for 14-16 hours at 37° C. while shaking at 250 RPM.

On day three, sample plates of E. coli colonies (roughly 100 microns in diameter) were prepared. First several LB agar plates (LB broth, 1.5% Agar, 100 μg/mL Ampicillin) were prewarmed for˜one hour at 37° C. Next 1:500 dilutions were made of the two overnight cultures described above using sterile LB broth. Part of the two dilutions were then removed and mixed to generate a mixture of fluorescent and non-fluorescent cells, generally 0.1% to 1% pGFPuv, the remainder being non-fluorescent Dummy#4. 50 μL of this mixture of cells was then spread on the pre-warmed LB agar plates. The plates were grown for seven hours at 37° C. in an incubator and then stored at 4° C. in a refrigerator for scanning the following day.

On day four, a fluorescence image of each plate was recorded. National Instruments' Labview was used to direct plate scanning and correlate the stage controller's positional data with the fluorescence emission intensity. The scanning and imaging of the fluorescence on the plates was performed by using a 488 nm continuous wave argon ion laser as the excitation source. The laser was set to deliver 40 μW of light energy at the 10× microscope objective. The laser was scanned over the plate by moving a translation stage to which the plate is attached. Fluorescence photons were collected during this scanning by using the same objective lens using to focus the laser beam onto the plate. These photons were directed through a series of optics onto an Avalanche Photo-Diode (APD) using a dichroic beam splitter to separate the incoming laser light from the emission and spectral filters to limit detection to wavelengths above 500 nm. For maximum resolution the stage speed was set to 1 cm/s. The program Matlab was used to read the data file output by Labview and construct a positionally encoded fluorescent image of the E. coli colonies. The image was then analyzed with Malab to determine the XY coordinates of the brightest colonies in the scan field. The kinetic mount holding the sample plate was then removed and the plate was exposed to light from a UV transilluminator (254 nm) for 10 s. Next the kinetic mount was placed back on the microscope stage and the coordinates of the desired colonies were programmed into a Labview program which directed the stage to move to those coordinates repeatedly (cycling between positions), using a mechanical shutter to selectively expose the colonies at the desired positions to 2 μW of 400 nm light from a Millenia Tsunami Ti-Saphire laser. This wavelength of light resulted in photoreactivation. The photoreactivation was carried out over ˜20 minutes. The plates were then grown in the dark at 37° C. overnight. Colonies targeted for photoreactivation grew normally while the other colonies' growth was retarded or abolished.

While the methods and systems have been described in connection with preferred embodiments and specific examples, it is not intended that the scope be limited to the particular embodiments or examples set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications are referenced. The disclosures of these publications in their entireties are hereby incorporated by reference into this application in order to more fully describe the state of the art to which the disclosed pertains.

It will be apparent to those skilled in the art that various modifications and variations can be made in the present methods and systems without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method for positive cell selection comprising:
providing a substrate with multiple locations;
immobilizing one or more cells comprising a photosensitive repair system and capable of exhibiting an imagable property when excited, indicating a desirable cell, either onto or within said substrate;
detecting the desirable cells by applying an excitation light source to the substrate to excite the imagable property of at least one of the one or more cells;
recording locations containing the desirable cells and locations not containing the desirable cells;
generating a repair image from the locations of the desirable cells;
applying a lethal light source to the substrate wherein the lethal light source is lethal to the cells, if the cells are not exposed to a repair light source capable of activating the photosensitive repair systems of the cells scanned; and
projecting a repair light source according to the repair image onto the substrate, the projection having light and dark portions wherein the light portions are capable of activating the photosensitive repair systems of the cells projected upon.

2. The method of claim 1, wherein the lethal light source is ultraviolet light.

3. The method of claim 1, wherein the repair light is visible light.

4. The method of claim 1, wherein the repair light is light having a wavelength from about 350 to about 700 nm.

5. The method of claim 1, wherein the imagable property is fluorescence.

6. The method of claim 1, further comprising determining at least one of the following: an excited state lifetime, an amplitude, a polarization, and a spectrum of the imagable property for at least one of the one or more cells.

7. The method of claim 6, wherein the repair image is further generated from at least one of an excited state lifetime, an amplitude, a polarization, and a spectrum.

8. The method of claim 1, wherein the imagable property of the cells is sensed and recorded by a charge couple device based (CCD) camera.

9. The method of claim 1, wherein a computer-controlled projection device is used to project the light onto the cells exhibiting the desired characteristic of the imagable property.

10. The method of claim 1, wherein the computer-controlled projection device is a digital light processor.

11. A method for positive cell selection comprising:
providing a substrate with multiple locations;
immobilizing one or more cells comprising a photosensitive repair system and capable of exhibiting an imagable property when excited, indicating a desirable cell, either onto or within said substrate;
detecting the desirable cells by scanning a first light beam across the substrate to excite the imagable property of at least one of the one or more cells;
recording locations containing the desirable cells and locations not containing the desirable cells;
generating a repair image from the locations of the desirable cells;
scanning a second light beam across the substrate wherein the second light beam is lethal to the cells, if the cells are not exposed to a light beam capable of activating the photosensitive repair systems of the cells scanned; and
scanning a third light beam, according to the repair image, wherein the third light beam is capable of activating the photosensitive repair systems of the cells scanned.

12. The method of claim 11, wherein the first light beam and the third light beam are from a common light source.

13. The method of claim 11, wherein the second light beam is from an ultraviolet light source.

14. The method of claim 11, wherein the third light source is from a visible light source.

15. The method of claim 11, wherein the first light beam is configured to perform two photon excitation of the repair system.

16. The method of claim 11, wherein the imagable property is fluorescence.

17. The method of claim 11, further comprising determining at least one of an excited state lifetime, an amplitude, a polarization, and a spectrum of the imagable property for at least one of the one or more cells.

18. The method of claim 17, wherein the repair image is further generated from at least one of an excited state lifetime, an amplitude, a polarization, and a spectrum.

19. An apparatus for positive cell selection comprising:
a means for holding a sample of cells comprising a photosensitive repair system and capable of exhibiting an imagable property when excited, indicating a desirable cell;
an excitation light source capable of exciting the imagable property;
a means for detecting the location of the desirable cells;
a means for generating a repair image representative of the locations of the desirable cells;
a lethal light source capable of killing the sample of cells, if not exposed to a light source capable of activating the photosensitive repair systems of the cells illuminated; and
a repair light source capable of activating the photosensitive repair systems according to the repair image.

20. The apparatus of claim 19, wherein the lethal light source is an ultraviolet light source.

21. The apparatus of claim 19, wherein the repair light is visible light.

22. The apparatus of claim 19, wherein the repair light source is light having a wavelength from about 350 to about 700 nm.

23. The apparatus of claim 19, wherein the imagable property is fluorescence.

24. The apparatus of claim 19, further comprising a means for determining at least one of an excited state lifetime, an amplitude, a polarization, and a spectrum of the imagable property for at least one of the one or more cells.

25. The apparatus of claim 24, wherein the repair image is further generated from at least one of an excited state lifetime, an amplitude, a polarization, and a spectrum.

26. The apparatus of claim 19, wherein the means for detecting is selected from the group consisting of:
a charge couple device based (CCD) camera;
a streak camera;
an avalanche photodiode; and
a photomultiplier tube.

27. The apparatus of claim 19, wherein the repair light source is configured to emit radiation such that two-photon absorption occurs.

28. The apparatus of claim 19, wherein the repair light source is a computer-controlled projection device.

29. The apparatus of claim 28, wherein the computer-controlled projection device is a digital light processor.

30. The apparatus of claim 19, wherein the repair light source is a laser.
